(19) United States
(12) Patent Application Publication
(10) Pub. No.: US 2020/0179258 A1
(43) Pub. Date: Jun. 11, 2020

(54) COMPOSITIONS, USES AND METHODS FOR TREATING OR PREVENTING DENTAL CARIES

(71) Applicant: APOLLONIA HEALTH INC., HAMILTON (CA)

(72) Inventors: Joon Kim, Hamilton (CA); Dennis Gerard Cvitkovitch, Oakville (CA)

(21) Appl. No.: 16/545,807

(22) Filed: Aug. 20, 2019

Related U.S. Application Data
(63) Continuation of application No. 14/647,380, filed on May 26, 2015, now abandoned.
(60) Provisional application No. 61/729,804, filed on Nov. 26, 2012.

(51) Int. Cl. A61K 8/60; A61Q 11/00; A61K 8/73; A61K 8/49; A61K 8/362; A61K 8/34; A61K 8/19; A61K 31/047; A61K 9/00; A61K 47/10; A61K 33/00; A61K 47/36; A61K 31/194

(52) U.S. Cl. CPC A61K 8/60; A61K 2800/30; A61K 8/733; A61K 8/4993; A61K 8/362; A61K 8/345; A61K 8/19; A61K 31/047; A61K 9/0095; A61K 47/10; A61K 33/00; A61K 47/36; A61K 31/194; A61K 2800/92; A61K 2800/592; A61Q 11/00

(57) ABSTRACT

The present invention provides compositions, uses and methods thereof, for inhibiting the growth of caries-causing bacteria. The composition comprises xylitol, sodium citrate, sodium bicarbonate, anionic polymers and acceptable carrier materials. The compositions can tend to be used for treating or preventing a condition caused by caries-causing bacteria The compositions can further tend to be used in patients such as children, adolescents or patients suffering from a heightened susceptibility to toxic substances.

### RELATED APPLICATIONS

This application claims priority from U.S. Provisional Application No. 61/729,804 filed Nov. 26, 2013, the contents of which are herein incorporated by reference.

### FIELD OF INVENTION

This invention relates to compositions and methods for treating or preventing dental caries. More particularly, the present invention relates to compositions and methods for inhibiting the growth of caries-causing bacteria.

### BACKGROUND

Dental caries is an undesirable condition of the oral cavity. Dental caries can cause extensive crown mutilations, bacterial disorders of the periapical tissues, or even loss of the affected dental elements. It is characterized by demineralization of the dental enamel and dentin in various stages of progress until it affects the pulp space. When the lesion passes beyond the enamel-dentin border, a phlogistic reaction of the pulp tissues is observed, with the formation of reaction dentin in some cases.

Dental caries is caused by bacteria that colonize in the mouth and produce acid which eventually leads to dissolution of the tooth enamel. These acidogenic bacteria include Streptococcus mutans (S. mutans), Streptococcus sobrinus, and Lactobacili, amongst others. These bacteria are resident of the biofilm environment of dental plaque, a matrix of bacteria and extracellular material that adheres to the tooth surface. Under appropriate conditions, populations of these bacteria will rise and the pH of the surrounding plaque will drop. These bacteria, being among the most acid tolerant organisms residing in dental plaque, will increase in numbers in this acidic environment and eventually become a dominant member of the plaque community. Once attached, the bacteria metabolize six-carbon dietary sugars, such as glucose, sucrose, and fructose into lactic acid. This situation eventually leads to dissolution of the tooth enamel, resulting in the development of dental caries. Since there is a strong correlation between the proportion of S. mutans in dental plaque or in saliva relative to other bacterial species and the presence or risk of future outbreaks of dental caries, S. mutans in plaque or saliva may serve as an index for both dental caries activity state and dental caries risk or susceptibility. These indices play an increasingly important role in the diagnosis and treatment of dental caries.

Recently it has become clear that dental plaque actually consists of hundreds of different bacterial taxa. Most of these bacteria exist on the surface of teeth in heterogeneous communities called plaque or biofilms. The mouth thus acts as a reservoir for these bacteria. While most of these bacteria are commensal, meaning they fail to adversely affect the human host, others are pathogenic and can cause tooth decay. Moreover these pathogenic bacteria have been found to cause a life-threatening disease called endocarditis.

S. mutans has also been found to contribute to infective endocarditis. Infective endocarditis is a potentially lethal infection of both native (normal) and artificial heart valves, and if left untreated can be fatal. S. mutans forms biofilms on the surface of these valves, and are typically a mixed community of a variety of pathogenic bacteria. Since these bacteria find their way to the valves via the blood stream and the blood stream is typically aseptic, there are usually few opportunities for bacteria to cause these infections. One exception is when pathogenic bacteria, like S. mutans, enter the blood stream during dental procedures. Hence, the probability of infection is directly related to the reservoir of infectious bacteria found in the oral cavity.

Previous efforts toward the correction of dental caries have revolved around the use of the standard toothbrush to remove dental plaque. Also in widespread use today are electric brushes, floss and adjuncts such as proxy brushes. In addition, numerous toothpastes and mouth rinses containing various supplements are touted as aids in the prevention of dental caries. For example, fluoride is commonly sold as a product for slowing the process of dental decay. However, the efficacy of such methods of treating or preventing dental caries is questionable. Dental plaque can only partially be removed from the oral cavity, even when a demanding regimen of oral hygiene that may include flossing, brushing and regular visits to a dentist is followed. In addition, many toothpastes and mouth rinses contain toxic supplements, such as fluoride and triclosan, which can be toxic to very young children.

In 2010, the US Surgeon General stated that dental caries is the most common chronic disease in children; it is five times more frequent than asthma and seven times more frequent than hay fever. The National Health and Nutrition Examination Survey conducted from 1999 to 2004 found that close to one in two children between the ages of 2 and 11 in the United States has at least one dental cavity in his or her primary teeth. (See United States, National Health and Nutrition Examination Survey, 1999-2004). The American Academy of Pediatric Dentistry (AAPD) guidelines for reducing caries incidence rates in infant and adolescent dental patients recommends fluoridation in addition to periodic professional dental services. Recommendations for pediatric fluoridation include a “smear” amount of fluoridated toothpaste for children under the age of 2 and a “pea-size” amount for children aged 2 to 5. In weighing the risk-benefit profile of using fluoride in children, the AAPD considered mild fluorosis (excessive fluorine consumption marked by visible white spots on teeth) to be an insignificant health hazard compared to prevention of the serious health risks of dental disease (See American Academy Of Pediatric Dentistry, Guideline on Infant Oral Health Care, 2012, Reference Manual V 35/NO 6 13/14).

Fluoride is also widely used in oral hygiene products. For example, many toothpastes and mouth rinses contain fluoride. In addition, many cities in Canada and the US have practiced public water fluoridation for more than 60 years.

However, the systemic use of fluoride to prevent cavities is controversial due to a potential correlation between cancer and fluoride. According to The American Cancer Society, osteosarcoma (cancer of the bone) is a rare form of cancer affecting 400 children and teens every year in the US. A number of long-term animal and human study data has been collected, and in certain studies, correlations have been found between higher levels of fluoride in drinking water and elevated incidences of osteosarcoma in both mice and humans, including children (See Levy M, Leclerc B S. Fluoride in drinking water and osteosarcoma incidence rates in the continental United States among children and adolescents. Cancer Epidemiol. 2012:36: 83-88; See Comber H, Deady S, Montgomery E, Gavin A. Drinking water fluoridation and osteosarcoma incidence on the island of Ireland. Cancer Causes Control. 2011;22:919-924).

In its 2011 publication entitled Guideline on Xylitol Use in Caries Prevention, the AAPD recommended using xylitol to prevent caries in moderate to high-risk pediatric patients. The guideline recommends administering xylitol dosages of between 3 and 8 grams per day in divided doses to prevent caries in children of all ages. Currently available oral hygiene products and inventions, however, even if they do contain xylitol, tend to also include fluoride and alcohol and other toxic substances. Accordingly these substances can tend to be harmful when ingested, and thus particularly unsuitable for dental caries prevention and treating compositions for children and other vulnerable groups.

Despite many previous efforts to formulate a treatment to treat or prevent dental caries, there is still a need for an effective treatment for dental caries.

There is a need for improved oral compositions for preventing and treating dental caries that do not contain fluoride. There is also a need for providing oral compositions for preventing and treating dental caries that are suitable for use by children and other caries-vulnerable groups in the general population. Further, there is a need for providing compositions that tend to maintain a high pH in the oral cavity environment, as dental caries causing bacteria thrive in acidic environments. Additionally, there is a need for providing compositions for treating and preventing dental caries that are longer lasting, and which do not contain toxic substances that are harmful when ingested.

### SUMMARY OF THE INVENTION

In an aspect of the present invention there is provided a composition for inhibiting the growth of caries-causing bacteria, comprising sodium bicarbonate, sodium citrate, at least one anionic polymer and xylitol. In some embodiments the sodium bicarbonate concentration is between 0.1% to 1.5% w/w, in other embodiments, the sodium citrate concentration is between 0.1% to 1% w/w, and in still further embodiments, either or both the sodium bicarbonate and sodium citrate concentration is 0.5% w/w. In still further embodiments, the sodium bicarbonate to sodium citrate ratio is between 1:1 to 2:1.

In some embodiments the anionic polymer is sodium alginate, and in further embodiments the sodium alginate concentration is between 0.05% to 0.5% w/w, or is 0.1% w/w. In some embodiments the xylitol concentration is between 15% to 40% w/w, or is 30% w/w.

In some aspects of the instant invention, the composition further comprises at least one excipient. In some embodiments, the excipient is a binder, a lubricant, a disintegrant, a suspending agent, an absorbent, a preservative, a surfactant, a colorant, a suspending agent, water, glycerin, a flavouring agent, an emulsifier, or a polyglycitol syrup.

In some embodiments wherein the composition comprises water, the water concentration can tend to be between 35% to 85% w/w, between 50% to 75% w/w, or is 58.75% w/w. In some embodiments wherein the composition comprises glycerin, the glycerin concentration can tend to be between 10% to 30% w/w, between 15% to 25% w/w or is 10%. In some embodiments wherein the composition comprises polyglycitol syrup, the polyglycitol concentration can tend to be between 0% to 15% w/w or between 5% to 10% w/w.

In some aspects of the invention, the composition further comprises at least one natural flavouring agent. The natural flavouring agent concentration can tend to be less than 0.1% w/w or can be 0.05% w/w. In some embodiments the natural flavouring agent is a natural fruit flavor such as lemon.

In some aspects of the invention, the composition comprises Polysorbate 20. In some embodiments wherein the composition comprises Polysorbate 20, the Polysorbate 20 concentration can tend to be between 0.01% to 0.1% w/w, or can be 0.1%.

In some aspects of the invention, the composition comprises an anti-microbial agent. In some embodiments wherein the composition comprises methylparaban as an anti-microbial agent, the methylparaban concentration can tend to be less than 0.1% w/w.

In some aspects of the invention, the composition can tend to be an oral formulation such as a liquid, a mouthwash, a dentifrice, a varnish, a gel, a food product, a confectionary, an ice cream, a chewing gum, a syrup, a cream, a tablet, a caplet, a capsule, a chewable tablet, a quick dissolve tablet, an effervescent tablet, a hard gelatin capsule, a soft gelatin capsule, a powder, or a liquid suspension.

In another aspect of the present invention there is provided a use of the composition for treating or preventing caries in a patient. In some aspects the composition can be used by children, adolescents or patient's suffering from a heightened susceptibility to toxic substances.

In further embodiments of the present invention there are provided uses of the composition for preparing a formulation for treating or preventing caries, treating or preventing a condition caused by caries-causing bacteria, preparing a formulation for treating or preventing a condition caused by caries-causing bacteria, inhibiting the growth of a caries-causing bacteria, and preparing a formulation for inhibiting the growth of a caries-causing bacteria. In some embodiments the caries-causing bacteria are Streptococcus mutans, Streptococcus sobrinus, or Lactobacili.

### BRIEF DESCRIPTION OF THE DRAWINGS

For a better understanding of embodiments of the system and methods described herein, and to show more clearly how they may be carried into effect, reference will be made by way of example, to the accompanying drawings in which:

FIG. 1 shows the pH of the culture medium after biofilms were incubated with various test treatments for 1 minute,

FIG. 2 shows the drop in pH of the culture medium 6 hours after biofilms were incubated with various test treatments for 1 minute.

FIG. 3 shows the drop in pH of the culture medium 8 hours after biofilms were incubated with various test treatments for 1 minute.

FIG. 4 shows the survival of S. mutans cells 1 hour after a 1 minute incubation with various test treatments, wherein survival was measured using a Presto Blue™ assay (*p<0.05).

FIG. 5 shows the survival of S. mutans cells 1 hour after a 1 minute incubation with various test treatments, wherein survival was measured using a colony forming unit (CFU) assay (*p<0.05).

### DETAILED DESCRIPTION

The present invention has been described with regard to specific embodiments. However, it will be obvious to persons skilled in the art that a number of variants and modifications can be made without departing from the scope of the invention as described herein.

In particular, numerous specific details are set forth in order to provide a thorough understanding of the embodiments described herein. However, it will be understood by those of ordinary skill in the art that the embodiments described herein may be practiced without these specific details. In other instances, well-known methods, procedures, and components have not been described in detail so as not to obscure the embodiments described herein. Furthermore, this description is not to be considered as limiting the scope of the embodiments described herein in any way, but rather as merely describing the implementation of the various embodiments described herein.

The compositions for treating and preventing dental caries described herein are particularly useful for administration to children and other vulnerable groups. There are presently no commercially available formulations for these consumers that are specifically formulated to improve cavity prevention and treatment, that include only Generally Recognized As Safe (GRAS) ingredients, that increase pH for improved mineralization, and that increase efficacy duration times.

As children tend to have weaker immune systems than adults, parents generally avoid even small risks with respect to the health of their children. The present invention tends to provide caries prevention solutions to the most caries-vulnerable age group, children aged 3 to 12, by increasing oral pH using bio-adhesive materials to provide long lasting efficacy and safe ingredients. Persons of skill will appreciate, however, that the compositions described herein can be suitable for use by other populations, include adult and geriatric populations.

In an embodiment of the present invention, there is provided a composition for treating and preventing dental caries which tends to increase the pH of the dental environment. Persons of skill will appreciate that dental caries occur due to the production of acid by bacteria that colonize in the mouth, leading to dissolution of the tooth enamel. The pH balance of the inside of the mouth determines how fast demineralization (the process of losing mineral, mainly calcium, from tooth enamel) and remineralization (the process of gaining mineral back to tooth enamel) occur to determine the rate of caries occurrence in one's mouth. When the pH range falls below 5.5, demineralization mainly occurs, and if the low pH is maintained for a prolonged period of time, the cavity occurrence increases drastically. Currently available oral hygiene products, however, tend to have a low pH.

The present inventions provide hygiene products that increase oral pH to promote remineralization and prevent demineralization of tooth enamel. In some embodiments the composition comprises sodium bicarbonate. In still further embodiments, the composition comprises sodium citrate. These ingredients tend to maintain a high pH in the oral cavity environment. When these two salts are combined in an aqueous solution, the anions, citrate and bicarbonate, act as alkalinizing agents to increase the pH of the solution, thereby providing a composition that tends to increase the pH of a dental environment when present in the oral cavities of the mouth.

In some embodiments, Sodium bicarbonate content can range between 0.1% to 1.5% w/w. In still further embodiments. Sodium citrate can range between 0.1% to 1% w/w. In alternative embodiments, the ratio of sodium bicarbonate and sodium citrate tends to be between 1 to 1 and 2 to 1.

Whether the actives in mouthwash or toothpaste are anti-bacterial or fluoride-like enamel protection agents, persons of skill will appreciate that a general recommendation from dental healthcare practitioners and manufacturers is to refrain from eating or drinking for 30 minutes after rinsing or brushing with standard cavity fighting agents. Eliminating the 30 minute waiting time would be advantageous to the general population, including active adults, busy parents and impatient children.

Embodiments of the present invention provide compositions for controlled release of dental caries prevention and treatment ingredients. In some embodiments, the compositions comprise anionic polymers to absorb the active ingredients and then release the active ingredients as hydration occurs. In further embodiments the compositions comprise sodium alginate, naturally found in the cell walls of brown algae, to control the release of other chemicals in the composition. Such controlled release substances can tend to reduce waiting times after application of the compositions described herein.

In some embodiments, the natural bio-adhesive and controlled-release material, sodium alginate, can tend to be used to prolong the beneficial effects of composition ingredients, such as xylitol, sodium bicarbonate and sodium citrate in the oral cavity. Sodium alginate content can tend to be between 0.05% and 0.5% w/w.

Embodiments of the presently claimed compositions are comprised of ingredients derived from natural sources or ingredients that are Generally Recognized As Safe (GRAS) materials. These safe ingredients can tend to be used in anticipation of accidental ingestion by consumers, especially children.

Despite the lack of unequivocal confirmation that fluoride is harmless, because of its known health benefits in caries prevention, fluoride is widely used in oral hygiene products. Other known formulations include water-soluble calcium salt to prevent or treat cavities, although persons of skill will appreciate that the use of calcium salt can tend to cause adverse effects. These adverse effects include calcium overdose, especially if the intended user group is children who drink homogenized milk or consume calcium-rich food. Further known compositions for preventing and treating caries tend to include alcohol, a substance that acts as a well-known depressant when consumed.

Embodiments of the present invention tend not to contain alcohol, fluoride or other chemicals deemed a risk when ingested. The presently claimed invention provides effective caries prevention without using harmful chemical agents that could pose health risks to users if accidentally ingested.

Embodiments of the present invention comprise xylitol, a sugar alcohol naturally found in many foods such as berries, oats, and mushrooms. Xylitol can tend to reduce the ability of caries-causing bacteria to adhere to the cell surface and to inhibit the bacteria from metabolizing six-carbon dietary sugars into lactic acid.

In a preferred embodiment, the composition can tend to comprise between 3 to 8 grams of xylitol given in divided dosages. The compositions described herein can tend to comprise between 15% to 40% w/w xylitol, which tends to be required and tends to be important to prevent cavities.

The compositions described herein can be made in a variety of forms, such as the following compositions: mouthwash, dentifrice, varnish, gel, confectionary, ice cream, chewing gum, syrup, cream, and the like. In other embodiments, the compositions can be made in other forms such as a tablet, a caplet, a capsule, chewable tablet, a quick dissolve tablet, an effervescent tablet, a hard gelatin capsule, a soft gelatin capsule, a powder, a liquid suspension, and other types of food products. One skilled in the art would recognize there are also other viable ways for delivering the composition to a user. In an embodiment, the composition is in a liquid dosage form; and in further embodiments, the composition is in a mouthwash form.

Furthermore, these compositions can be made using conventional equipment and techniques known in the art. When preparing dosage forms incorporating the compositions of the invention, the ingredients are normally blended with conventional excipients such as binders, lubricants, disintegrants, suspending agents, absorbents, preservatives, surfactants, colorants and suspending agents.

Additional carrier materials can tend to be included, for example water, glycerin, flavouring agents and emulsifyiers. Persons of skill in the art will appreciate that other ingredients can be included.

In embodiments of the present invention water can tend to be present in the range of between 35% to 85% w/w. In further embodiments, water can tend to be between 50% to 75% w/w. In embodiments of the present invention wherein compositions are in the form of mouthwash, water can tend to be the main solvent.

Glycerin, with or without polyglycitol syrup, can be used to add sweetness, taste, viscosity and texture to embodiments of the present invention. The range for glycerin content can tend to be between 10% and 30% w/w. In further embodiments, the glycerin content can tend to be between 15% and 25% w/w. The range for polyglycitol syrup can tend to be between 0% and 15% w/w in some embodiments, and between 5% and 10% w/w in further embodiments.

Natural fruit flavours, and not artificial flavours, can be used in some embodiments to improve smell and taste of the compositions. Natural flavouring agents can tend to be present at a concentration of less than 0.1% w/w, possibly with a combination of different flavours.

An emulsifier, Polysorbate 20, can tend to be included in some embodiments to disperse the flavouring agents. For example, the composition may contain between 0.01% to 0.1% w/w of Polysorbate 20.

In some embodiments, the present invention is formulated to prevent microbial contamination by regulating water activity (aw), thereby reducing dependence on preservative agents. The naturally-occurring anti-microbial agent, methyparaban, can be used in some embodiments of the invention as a preservative to prevent undesired microbial growth. In alternative embodiments, the composition can comprise methylparaban in amounts of less than 0.1% w/w.

Use of xylitol in combination with sodium citrate, sodium bicarbonate and sodium alginate tends to provide a synergistic effect. As discussed in the examples below, it has been observed that in tests using S. mutans cultures, a composition containing xylitol, sodium citrate, sodium bicarbonate and sodium alginate results in an increase in the pH of the culture medium and a decrease in S. mutans viability as compared with xylitol, or any of the other active components, alone.

### EXAMPLES

### Example 1—Composition for inhibiting Dental Caries (Xyncal 8.3)

An example of an embodiment of a composition for inhibiting dental caries, referred to throughout this application is:

| Ingredient | % w/w | grams per 5 g dose |
|---|---|---|
| Water | 58.75 | 2.938 |
| Glycerin | 10.00 | 0.500 |
| Xylitol | 30.00 | 1.500 |
| Sodium Bicarbonate | 0.50 | 0.025 |
| Sodium Citrate | 0.50 | 0.025 |
| Sodium Alginate | 0.10 | 0.005 |
| Flavor | 0.05 | 0.003 |
| Polysorbate 20 | 0.10 | 0.005 |

The composition of Example 1 will herein be referred to as Xyncal 8.3.

### Example 2—Potential Product Specification

A sample product specification for an embodiment of the present invention is listed below:

| Test | Specification |
|---|---|
| Solids, % w/w by hand refractometer (° brix) | 25.0 +/− 2.0 |
| pH, as is | 08.3 +/− 0.3 |
| Colour | Colourless |
| Appearance | Clear liquid |
| Odour | Lemon |
| Flavour | Sweet, lemon flavour |
| Mouth feel and texture | Slight body |
| APC (aerobic plate count). | <20 |
| Y&M (yeasts and moulds) | |

### Example 3—Preparing S. mutans Biofilms

### Bacteria Strain Used

Amongst acidogenic bacteria, S. mutans is the principal etiological agent of caries in humans. Since there is a strong correlation between the proportion of S. mutans in dental plaque or in saliva relative to other bacterial species and the presence or risk of future outbreaks of dental caries, S. mutans in plaque or saliva may serve as an index for both dental caries activity state and dental caries risk or susceptibility. S. mutans UA159 strain was used in this Example.

### Biofilm Preparation

On the first day, biofilm growth was initiated by inoculating 10mL of 100% Todd-Hewitt broth (THYE) with 0.3% yeast extract at a pH of 7.0 with UA159 to prepare overnight cultures. The samples were incubated in closed screw-cap tubes without agitation at 37° C. in an anaerobic chamber (5% CO2),

On the second day, 1:100 dilutions were prepared by mixing 20 μl of the overnight UA159 cultures with ¼ THYE with 0.075% yeast extract supplemented with 0.1% sucrose to reach a final volume of 2 mL. The dilutions were then placed into the wells of 12-well plates. The cultures were incubated at 37° C. for 18-24 hours without agitation in an anaerobic chamber (5% CO2).

Incubation with Test Treatments

On the third day, the 2 mL of medium was removed from each well. 1.0 mL of test treatment solution, as described below, was added to each well, after which the wells were incubated for 1 minute on the bench at room temperature. Next, the test treatment solutions were removed, and 3.0 mL of undiluted THYE with 0.3% yeast extract and no sucrose was added to each well. If an internal pH control was required, fresh THYE was also added to two un-inoculated wells. During these steps, care was taken to avoid disrupting the biofilm.

The following test treatments were used:

Water
Xyncal 8.3
Xylitol [1.5 g/5 ml] in water
Sodium bicarbonate [0.5 g/100 ml] in water
Sodium citrate dihydrate [0.5 g/100 ml] in water
Sodium Alginate [0.1 g/100 ml] in water
Listerine ™ Smart Rinse ™
Listerine ™ Agent Cool Blue ™

### Example 4—Effect of Composition of Example 1 (Xyncal 8.3) on pH in Cultures Containing S. mutans

An in vitro assay was performed to determine whether the compositions of the present invention would increase the pH in culture media containing S. mutans as compared to control treatments.

First, biofilms were prepared and incubated with test treatments as described in Example 3. Immediately after 100% THYE with 0.3% yeast extract was added to the wells following incubation with the test treatments, the initial pH of the medium in each well was measured. Samples were incubated at 37° C. in an open air, bench-top incubator, and the pH of the medium was measured once an hour for the next 7-8 hours. Finally, the phi of the medium in each well was measured again the next morning.

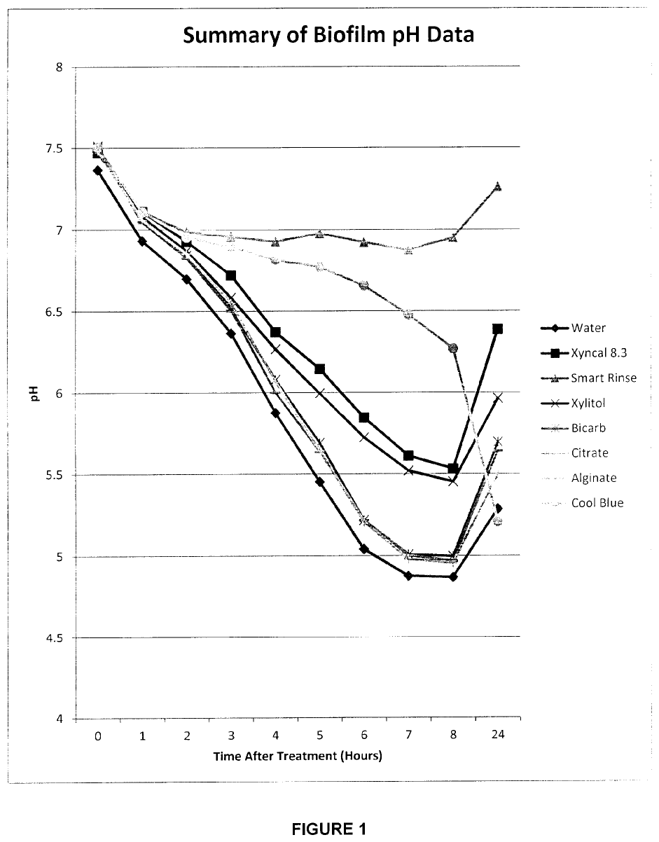

As shown in FIG. 1, a 1 minute incubation with Xyncal 8.3 resulted in the pH of the medium being above 5.5 for the duration of the assay. Xyncal 8.3 was able to maintain a higher pH than any of its individual components (xylitol, sodium citrate, sodium bicarbonate or sodium alginate) on their own, exhibiting an unexpected and synergistic effect. Listerine™ Smart Rinse™, however, was able to maintain the highest pH, which is believed to be due to the inclusion of the antiseptic Cetylpyridinium Chloride (CPC) in this rinse (which is also in Listerine™ Agent Cool Blue™). Since the rinse kills the biofilms, the pH of the medium does not remain as low.

As compared to the water control, the results from Xyncal 8.3 showed a statistically significant difference at 1, 6, 7, 8, and 24 hours (p<0.05); the results from Listerine™ Smart Rinse™ showed a statistically significant difference at all times (p<0.05); the results from xylitol showed a statistically significant difference at 1, 6, 7, and 8 hours (p<0.05); the results from sodium bicarbonate showed a statistically significant difference at 1 hour (p<0.05); and the results from Listerine™ Agent Cool Blue™ showed a statistically significant difference at 1 to 8 hours (p<0.05). The results from sodium citrate and sodium alginate showed no statistically significant difference at any time.

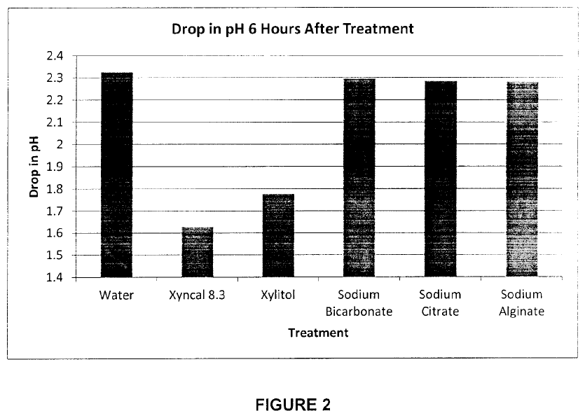

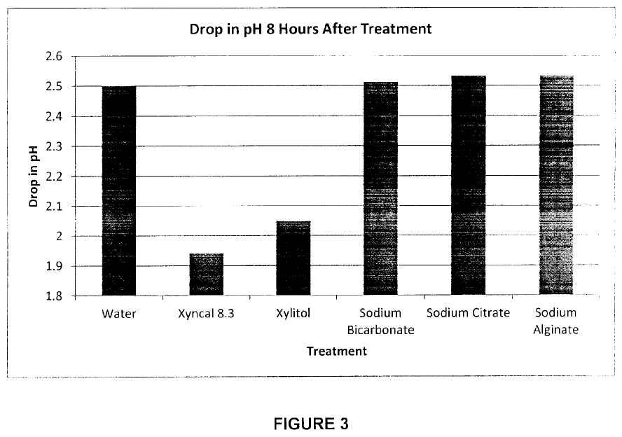

FIGS. 2 and 3 shows more clearly the synergistic effect that results when combining the components of Xyncal 8.3. FIG. 2 is a graph showing the drop, or difference in total pH value, 6 hours after incubation with the test treatments, i.e. the pH of the medium at 6 hours subtracted from the initial pH of the medium, wherein the test treatments are water, Xyncal 8.3. xylitol, sodium bicarbonate, sodium citrate, and sodium alginate. FIG. 3 is a similar graph showing the drop in pH 8 hours after treatment.

As can be seen from FIGS. 2 and 3, treating biofilms with any one of sodium bicarbonate, sodium citrate, or sodium alginate alone has little to no effect on the drop in pH; the drop is about equal to that of the control, water. Of the components of Xyncal 8.3, only xylitol is able to slow the pH drop. One would therefore expect that upon adding xylitol, sodium bicarbonate, sodium citrate, and sodium alginate together, the pH drop would be about as large as that for xylitol. However, as seen in FIGS. 2 and 3, the pH drop of the medium when treated with Xyncal 8.3 is actually lower than that for xylitol, showing synergy. Tables 1 to 8 below show exemplary pH data obtained upon performing the in vitro assays as described above.

TABLE 1

| test | | hours after treatment | | | | | | | | |
|---|---|---|---|---|---|---|---|---|---|---|
| treatment | | 0 | 1 | 2 | 3 | 4 | 5 | 6 | 7 | 8 |
| Water | Sample 1 | 7.32 | 6.9 | 6.48 | 5.8 | 4.86 | 4.68 | 4.68 | 4.67 | 4.75 |
| | Sample 2 | 7.34 | 6.91 | 6.69 | 6.48 | 6.09 | 5.57 | 5.04 | 4.93 | |
| | Sample 3 | 7.26 | 6.88 | 6.73 | 6.56 | 6.26 | 5.76 | 5.21 | 4.93 | 4.94 |
| | Sample 4 | 7.54 | 7.04 | 6.89 | 6.61 | 6.29 | 5.79 | 5.23 | 4.97 | 4.91 |
| | Average | 7.37 | 6.93 | 6.70 | 6.36 | 5.88 | 5.45 | 5.04 | 4.88 | 4.87 |
| | Standard | 0.12 | 0.07 | 0.17 | 0.38 | 0.68 | 0.52 | 0.25 | 0.14 | 0.10 |
| | Deviation | | | | | | | | | |

TABLE 2

| test | | hours after treatment | | | | | | | | |
|---|---|---|---|---|---|---|---|---|---|---|
| treatment | | 0 | 1 | 2 | 3 | 4 | 5 | 6 | 7 | 8 |
| Xyncal | Sample 1 | 7.46 | 7.12 | 6.81 | 6.53 | 6.01 | 5.76 | 5.49 | 5.2 | 5.11 |
| 8.3 | Sample 2 | 7.37 | 7.03 | 6.86 | 6.62 | 6.21 | 6 | 5.6 | 5.38 | |
| | Sample 3 | 7.5 | 7.12 | 7.02 | 6.86 | 6.64 | 6.42 | 6.13 | 5.93 | 5.72 |
| | Sample 4 | 7.56 | 7.18 | 7.02 | 6.87 | 6.62 | 6.4 | 6.16 | 5.93 | 5.76 |
| | Average | 7.47 | 7.11 | 6.93 | 6.72 | 6.37 | 6.15 | 5.85 | 5.61 | 5.53 |
| | Standard | 0.08 | 0.06 | 0.11 | 0.17 | 0.31 | 0.32 | 0.35 | 0.38 | 0.36 |
| | Deviation | | | | | | | | | |

TABLE 3

| test | | hours after treatment | | | | | | | | |
|---|---|---|---|---|---|---|---|---|---|---|
| treatment | | 0 | 1 | 2 | 3 | 4 | 5 | 6 | 7 | 8 |
| Listerine ™ | Sample 1 | 7.49 | 7.16 | 7.02 | 7.04 | 7.04 | 7.05 | 7 | 6.89 | 7.04 |
| Smart | Sample 2 | 7.34 | 7.02 | 6.89 | 6.83 | 6.8 | 6.91 | 6.81 | 6.78 | |
| Rinse ™ | Sample 3 | 7.59 | 7.13 | 7.01 | 6.97 | 6.92 | 6.98 | 6.92 | 6.9 | 6.89 |
| | Sample 4 | 7.57 | 7.13 | 7.03 | 6.99 | 6.95 | 6.97 | 6.96 | 6.93 | 6.92 |
| | Average | 7.50 | 7.11 | 6.99 | 6.96 | 6.93 | 6.98 | 6.92 | 6.88 | 6.95 |
| | Standard | 0.11 | 0.06 | 0.07 | 0.09 | 0.10 | 0.06 | 0.08 | 0.07 | 0.08 |
| | Deviation | | | | | | | | | |

TABLE 4

| test | | hours after treatment | | | | | | | | |
|---|---|---|---|---|---|---|---|---|---|---|
| treatment | | 0 | 1 | 2 | 3 | 4 | 5 | 6 | 7 | 8 |
| Xylitol | Sample 1 | 7.49 | 7.08 | 6.72 | 6.19 | 5.77 | 5.51 | 5.23 | 5.1 | 5.09 |
| | Sample 2 | 7.36 | 7 | 6.82 | 6.57 | 6.17 | 5.93 | 5.55 | 5.37 | |
| | Sample 3 | 7.58 | 7.12 | 6.97 | 6.77 | 6.55 | 6.25 | 6.05 | 5.78 | 5.58 |
| | Sample 4 | 7.57 | 7.12 | 6.97 | 6.8 | 6.57 | 6.29 | 6.06 | 5.82 | 5.68 |
| | Average | 7.50 | 7.08 | 6.87 | 6.58 | 6.27 | 6.00 | 5.72 | 5.52 | 5.45 |
| | Standard | 0.10 | 0.06 | 0.12 | 0.28 | 0.38 | 0.36 | 0.41 | 0.34 | 0.32 |
| | Deviation | | | | | | | | | |

TABLE 5

| test | | hours after treatment | | | | | | | | |
|---|---|---|---|---|---|---|---|---|---|---|
| treatment | | 0 | 1 | 2 | 3 | 4 | 5 | 6 | 7 | 8 |
| Sodium | Sample 1 | 7.48 | 7.09 | 6.67 | 5.85 | 5.08 | 5.03 | 5 | 5 | 5.06 |
| Bicar- | Sample 2 | 7.39 | 6.97 | 6.81 | 6.6 | 6.21 | 5.7 | 5.11 | 4.96 | |
| bonate | Sample 3 | 7.58 | 7.1 | 6.93 | 6.82 | 6.49 | 5.97 | 5.33 | 5.01 | 4.96 |
| | Sample 4 | 7.59 | 7.12 | 6.96 | 6.81 | 6.53 | 6.06 | 5.42 | 5.06 | 4.97 |
| | Average | 7.51 | 7.07 | 6.84 | 6.52 | 6.08 | 5.69 | 5.22 | 5.01 | 5.00 |
| | Standard | 0.09 | 0.07 | 0.13 | 0.46 | 0.68 | 0.47 | 0.19 | 0.04 | 0.06 |
| | Deviation | | | | | | | | | |

TABLE 6

| test | | hours after treatment | | | | | | | | |
|---|---|---|---|---|---|---|---|---|---|---|
| treatment | | 0 | 1 | 2 | 3 | 4 | 5 | 6 | 7 | 8 |
| Sodium | Sample 1 | 7.5 | 7.05 | 6.69 | 6.01 | 5.14 | 5.02 | 4.98 | 4.97 | 4.99 |
| Citrate | Sample 2 | 7.35 | 6.93 | 6.75 | 6.47 | 5.94 | 5.49 | 5.06 | 4.92 | |
| | Sample 3 | 7.57 | 7.09 | 6.91 | 6.76 | 6.44 | 5.96 | 5.36 | 5.04 | 4.95 |
| | Sample 4 | 7.58 | 7.12 | 6.95 | 6.77 | 6.48 | 6.07 | 5.46 | 5.07 | 4.96 |
| | Average | 7.50 | 7.05 | 6.83 | 6.50 | 6.00 | 5.64 | 5.22 | 5.00 | 4.97 |
| | Standard | 0.11 | 0.08 | 0.12 | 0.36 | 0.62 | 0.48 | 0.23 | 0.07 | 0.02 |
| | Deviation | | | | | | | | | |

TABLE 7

| test | | hours after treatment | | | | | | | | |
|---|---|---|---|---|---|---|---|---|---|---|
| treatment | | 0 | 1 | 2 | 3 | 4 | 5 | 6 | 7 | 8 |
| Sodium | Sample 1 | 7.45 | 7.09 | 6.74 | 6.14 | 5.24 | 4.99 | 4.96 | 4.97 | 4.96 |
| Alginate | Sample 2 | 7.36 | 6.93 | 6.77 | 6.48 | 6 | 5.47 | 5.02 | 4.92 | |
| | Sample 3 | 7.56 | 7.09 | 6.95 | 6.78 | 6.5 | 5.99 | 5.32 | 4.98 | 4.94 |
| | Sample 4 | 7.58 | 7.13 | 6.95 | 6.79 | 6.52 | 6.12 | 5.52 | 5.04 | 4.96 |
| | Average | 7.49 | 7.06 | 6.85 | 6.55 | 6.07 | 5.64 | 5.21 | 4.98 | 4.95 |
| | Standard | 0.10 | 0.09 | 0.11 | 0.31 | 0.60 | 0.52 | 0.26 | 0.05 | 0.01 |
| | Deviation | | | | | | | | | |

TABLE 8

| test | | hours after treatment | | | | | | | | |
|---|---|---|---|---|---|---|---|---|---|---|
| treatment | | 0 | 1 | 2 | 3 | 4 | 5 | 6 | 7 | 8 |
| Listerine ™ | Sample 1 | 7.47 | 7.13 | 6.94 | 6.85 | 6.72 | 6.53 | 6.3 | 5.89 | 5.49 |
| Agent | Sample 2 | 7.38 | 7.02 | 6.89 | 6.8 | 6.74 | 6.77 | 6.63 | 6.51 | |
| Cool | Sample 3 | 7.58 | 7.15 | 6.99 | 6.96 | 6.89 | 6.89 | 6.85 | 6.76 | 6.71 |
| Blue ™ | Sample 4 | 7.6 | 7.14 | 7 | 6.97 | 6.91 | 6.89 | 6.84 | 6.74 | 6.6 |
| | Average | 7.51 | 7.11 | 6.96 | 6.90 | 6.82 | 6.77 | 6.66 | 6.48 | 6.27 |
| | Standard | 0.10 | 0.06 | 0.05 | 0.08 | 0.10 | 0.17 | 0.26 | 0.41 | 0.67 |
| | Deviation | | | | | | | | | |

### Example 5—Effect of Xyncal 8.3 on S. mutans Growth and Biofilm Formation

An in vitro assay was performed to determine whether Xyncal 8.3 would show inhibitory effects on S. mutans growth and biofilm formation.

First, biofilms were prepared and incubated with test treatments as described in Example 3. immediately after undiluted THYE with 0.3% yeast extract was added to the wells following incubation with the test treatments, the samples were incubated for 1 hour at 37° C.

Next, one of two different assays was performed to test S. mutans growth and biofilm formation: a Presto Blue™ assay and a Colony Forming Unit (CFU) assay.

For the Presto Blue™ assay, the biofilms were resuspended in the media present in the wells. Next, the suspension was sonicated for 20 seconds to disrupt bacterial chains. The OD₆₀₀ value was measured and adjusted to an OD₆₀₀ value of 0.2. A 90 μL sample of the suspension was placed in the well of a 96-well plate after which 10 μL of Presto Blue™ reagent was added to the well. The samples were incubated at 37° C. for at least 10 minutes or until the samples began to turn pink. Finally, the samples were read in a plate reader at 570 nm and 600 nm and the 600 nm value was subtracted from the 570 nm value.

For the CFU assay, the biofilms were resuspended in the media present in the wells. The suspensions were sonicated for 20 seconds to disrupt bacterial chains. Next, the suspensions were serially diluted down to 10⁻⁷ by adding 20 μL of the cell suspension to 180 μL of Phosphate Buffered Saline (PBS). For each dilution, 20 μL of solution was plated on 100% THYE agar plates. After 24-48 hours, the CFUs were counted.

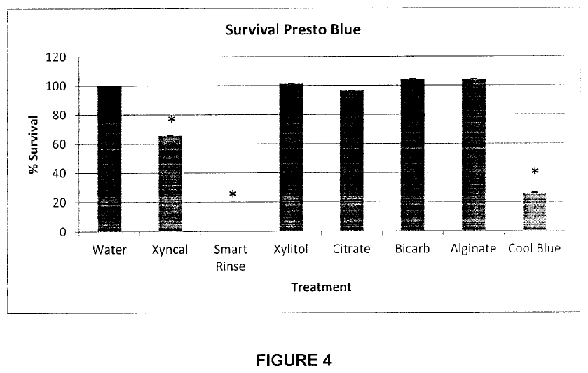

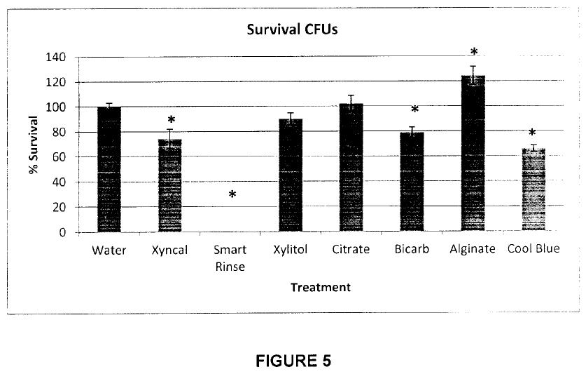

FIGS. 4 and 5 show the survival of S. mutans cells 1 hour after a 1 minute incubation with the test treatments, measured using the Presto Blue™ and CFU assays respectively. Both FIGS. 4 and 5 show that Xyncal 8.3 inhibits S. mutans growth and biofilm formation to a greater extent than any of its individual components (xylitol, sodium citrate, sodium bicarbonate or sodium alginate) on their own, exhibiting an unexpected and synergistic effect.

The survival assays correlate with, and explain, the data from Example 3. The increased inhibition of S. mutans growth and biofilm formation with Xyncal 8.3, as compared with its individual components (xylitol, sodiumitrate, sodium bicarbonate or sodium alginate), explain why Xyncal 8.3 was able to maintain a higher pH than any of its individual components. Listerine™ Smart Rinse™'s increased inhibition of S. mutans growth and biofilm formation is believed to be due to the inclusion of the antiseptic Cetylpyridinium Chloride (CPC) in this rinse (also in Listerine™ Agent Cool Blue™).

### Example 6—Clinical Study

A statistically non-powered and non-randomized clinical study was conducted involving eight consenting adults and one teenager and one toddler under their parents' supervision.

Other than the toddler, who swallowed without much rinsing, the participants rinsed their teeth once for 30 seconds with between 2.5 ml to 5 ml the mouthwash composition from Example 1. Nine participants chose to imbibe the mouthwash and one participant spat it out after 30 seconds of gurgling.

All 10 participants enjoyed the flavour, odour, feel and texture of the invention in the form of a mouthwash. No adverse effect was reported after 24 hours, one week, one month, and three months after imbibing or gurgling with the mouthwash.

Nine participants reported cleaner and more “pleasant” feelings after rinsing their teeth with the mouthwash compared to before rinsing, The “pleasant” feelings were described as cleaner, sweeter, and even better than brushing with toothpaste. They also reported that the “pleasant” feeling lasted between three hours and 12 hours. The toddler participant's parents reported that the toddler's “morning breath” had significantly improved and smelt pleasant in the morning.

The present invention has been described with regard to specific embodiments. However, it will be obvious to persons skilled in the art that a number of variants and modifications can be made without departing from the scope of the invention as described herein.

1. A composition for inhibiting the growth of caries-causing bacteria, comprising:
sodium bicarbonate;
sodium citrate;
at least one anionic polymer; and
xylitol.

2. The composition of claim 1, wherein the sodium bicarbonate concentration is between 0.1% to 1.5% w/w.

3. The composition of claim 1 or claim 2, wherein the sodium citrate concentration is between 0.1% to 1% w/w.

4. The composition of claim 1, wherein the sodium bicarbonate to sodium citrate ratio is between 1:1 to 2:1.

5. The composition of claim 1, wherein the sodium bicarbonate concentration is 0.5% w/w.

6. The composition of claim 1, wherein the sodium citrate concentration is 0.5% w/w.

7. The composition of claim 1, wherein the at least one anionic polymer is sodium alginate.

8. The composition of claim 7 wherein the sodium alginate concentration is between 0.05% to 0.5% w/w.

9. The composition of claim 8 wherein the sodium alginate concentration is 0.1% w/w.

10. The composition of claim 1, wherein the xylitol concentration is between 15% to 40% w/w.

11. The composition of claim 10 wherein the xylitol concentration is 30% w/w.

12. The composition of claim 1 further comprising at least one excipient.

13. The composition of claim 12 wherein the at least one excipient is one or more of a binder, a lubricant, a disintegrant, a suspending agent, an absorbent, a preservative, a surfactant, a colorant, a suspending agent, water, glycerin, a flavouring agent, an emulsifier, or polyglycitol syrup.

14. The composition of claim 13 wherein the water concentration is between 35% to 85% w/w.

15. The composition of claim 14 wherein the water concentration is between 50% to 75% w/w.

16. The composition of claim 15 wherein the water concentration is 58.75% w/w.

17. The composition of claim 13, wherein the glycerin concentration is between 10% to 30% w/w.

18. The composition of claim 17 wherein the glycerin concentration is between 15% to 25% w/w.

19. The composition of claim 17 wherein the glycerin concentration is 10%.

20. The composition of claim 13, wherein the polyglycitol syrup concentration is between 0% to 15% w/w.

21-43. (canceled)
